(19) United States
(12) Patent Application Publication
(10) Pub. No.: US 2011/0257631 A1
(43) Pub. Date: Oct. 20, 2011

(54) ACCELEROMETER BASED LASER LEVELING DEVICE

(75) Inventor: John Murdock Murphy, Jacksonville, FL (US)

(73) Assignee: Medtronic Xomed, Inc., Jacksonville, FL (US)

(21) Appl. No.: 12/763,335

(22) Filed: Apr. 20, 2010

(51) Int. Cl. A61M 27/00; G01C 9/00

(52) U.S. Cl. 604/540; 33/290

(57) ABSTRACT

A laser leveling device is disclosed for use with an external medical drainage system. The laser leveling device includes a housing enclosing an accelerometer and a laser module. At least one level indicator is electrically coupled to the accelerometer to provide an indication of whether the housing is level with respect to gravity.

### BACKGROUND

As well known in the medical arts, to relieve an undesirable accumulation of fluids from a part of the body, it is frequently necessary to provide a means for draining the fluid away from the body. Such is the case, for example, in the treatment of hydrocephalus, an ailment usually afflicting infants or children in which fluids which ought to drain away accumulate within the skull and thereby exert extreme pressure and skull deforming forces.

In treating hydrocephalus, cerebrospinal fluid accumulated in the brain ventricles is drained away by a catheter inserted into the ventricle through the skull, and the catheter is connected to a tube which conducts the fluid away from the brain either to another part of the body or to an external source. External drain systems typically are attached to intravenous (IV) poles and include a mounting assembly having a pole clamp and a scale, and a drip assembly adjustably fastened to the mounting assembly. The drip assembly typically includes a fluid-receiving graduated cylinder which often empties into a disposable bag.

A zero reference point on the skull is usually found using a laser leveling device. In particular, a zero point on the scale is aligned with this zero reference point on the skull. Additionally, a zero reference stop cock having fluid valves is also aligned with the zero reference point and usually attached to the pole clamp. In order to control the flow of cerebrospinal fluid and maintain the proper pressure in the brain ventricle, the drip assembly is elevated or lowered along the scale to encourage or reduce a gravity or pressure flow from the brain ventricles into the cylinder and/or bag.

Current laser leveling devices include a laser enclosed within a housing and one or more leveling tubes that indicate whether the housing (and thus the laser) is level with respect to gravity. The housing is attached to the scale so that the zero point on the scale, the zero point reference stop cock and the laser are all in fixed relation (typically aligned). A user inspects the leveling tube to determine whether the housing of the laser leveling device is level and/or whether rotation of the housing is needed to level the device. After the housing is level, the drainage system and/or patient can be positioned so that the laser is aligned with the zero reference point on the skull.

### SUMMARY

Aspects of concepts presented herein relate to a laser leveling device for use with an external medical drainage system. The laser leveling device includes a housing enclosing an accelerometer and a laser module. At least one level indicator is electrically coupled to the accelerometer to provide an indication of whether the housing is level with respect to gravity. In one particular aspect, the housing of the laser leveling device is rotatably coupled to the drainage system through a mounting bracket. A power source can further be enclosed within the housing to selectively provide power to the accelerometer, the laser module and the level indicator. Additionally, a switch can be electrically coupled to the power source such that, upon actuation of the switch, the power source provides power to the accelerometer and the laser module for a predetermined period of time.

In another aspect, a drainage system includes a catheter configured to be fluidly coupled to a patient and a drainage control component mounted to a panel spaced apart from the patient. The drainage control component is fluidly coupled to the catheter. A drip assembly is slidably mounted to the panel for selective positioning with respect to the drainage control component and fluidly coupled to the drainage control component. Furthermore, a laser leveling device is mounted to the panel and includes a housing, an accelerometer, a laser module and a level indicator. The level indicator is electrically coupled to the accelerometer to indicate a relative position of the housing with respect to gravity.

In yet another aspect, a method for draining fluid from a patient includes fluidly coupling a catheter to the patient and fluidly connecting a drainage control component to the catheter. A laser leveling device is positioned in fixed relation to the drainage control component and includes a housing, an accelerometer, a laser module and a level indicator. The level indicator is electrically coupled to the accelerometer to indicate a relative position of the housing with respect to the gravity. The method also includes rotating the housing so as to be level with gravity and powering the laser module to form a laser beam. The laser beam is aligned with a reference point on the patient.

### BRIEF DESCRIPTION OF THE DRAWINGS

FIG. 1 is a schematic view of an external drainage system.

FIG. 2 is a rear perspective view of a portion of the external drainage system of FIG. 1.

FIG. 3 is a front view of a laser leveling device.

FIG. 4 is a rear perspective view of the laser leveling device of FIG. 3.

FIG. 5 is a top view of the laser leveling device of FIG. 3.

FIG. 6 is a schematic diagram of components housed in the laser leveling device.

### DETAILED DESCRIPTION

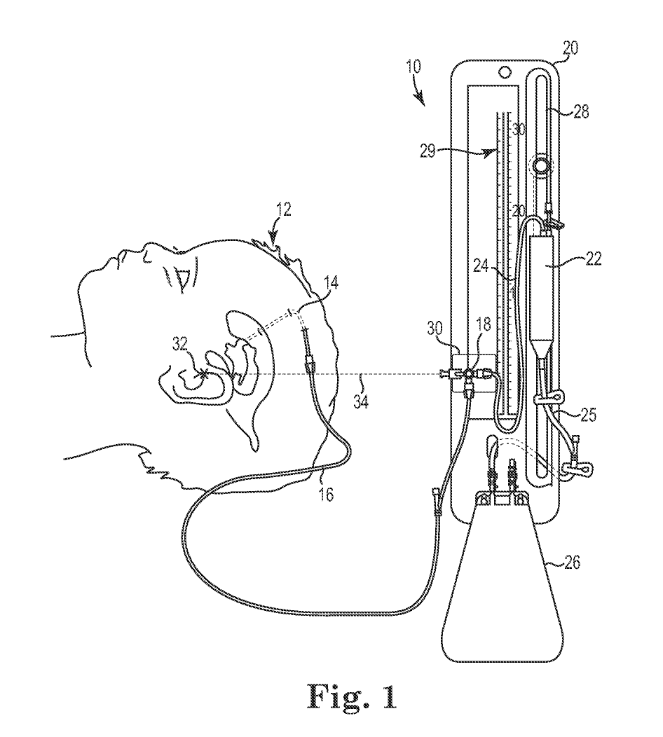

FIG. 1 is a schematic diagram of a drainage system 10 fluidly coupled to a patient 12. To fluidly couple the drainage system 10 to the patient 12, a catheter 14 is inserted into a skull of the patient to access a ventricle of the brain. Catheter 14 is fluidly connected to a first tubing segment 16 which leads to a drainage control component, herein embodied as a zero reference stop cock 18 mounted to a panel 20 of the drainage system 10. In particular, the zero reference stop cock 18 is mounted at a predetermined reference position on the panel 20. Zero reference stop cock 18 is also fluidly coupled to a drip assembly 22 through a second tubing segment 24. Fluid from drip assembly 22 further passes through a third tubing segment 25 to a collection bag 26. Drip assembly 22 is positioned along panel 20 within a slot 28 such that drip assembly 22 is positioned at a level relative to zero reference stop cock 18 as prescribed by a physician. Indicia 29 on the panel provide a reference for determining the relative difference between zero reference stop cock 18 and drip assembly 22. As fluid pressure builds up within a head of patient 12, fluid is drained from the catheter 14 to the collection bag 26 based on the relative position of zero reference stop cock 18 and drip assembly 22.

To establish a desired fluid pressure within the head of patient 12, a laser leveling device 30 is used to align zero reference stop cock 18 with a zero reference point 32 marked on patient 12. In one embodiment, device 30 is aligned with zero reference stop cock 18 and zero reference point 32 is located at a top of an ear of the patient 12. The laser leveling device 30 emits a laser beam 34 (schematically shown) and a position (i.e., height) of drainage system 10 and/or patient 12 is adjusted such that beam 34 is aligned with zero reference point 32. As discussed below, laser leveling device 30 includes at least one level indicator to insure that beam 34 is level with respect to gravity. In one embodiment, drainage system 10 is mounted to an IV pole (not shown) using a sliding mounting mechanism such that zero reference stop cock 18 and zero reference point 32 are positioned at the same height. One example mechanism for mounting drainage system 10 to a pole is shown and described in U.S. Pat. No. 6,540,727; the contents of which are hereby incorporated by reference in their entirety.

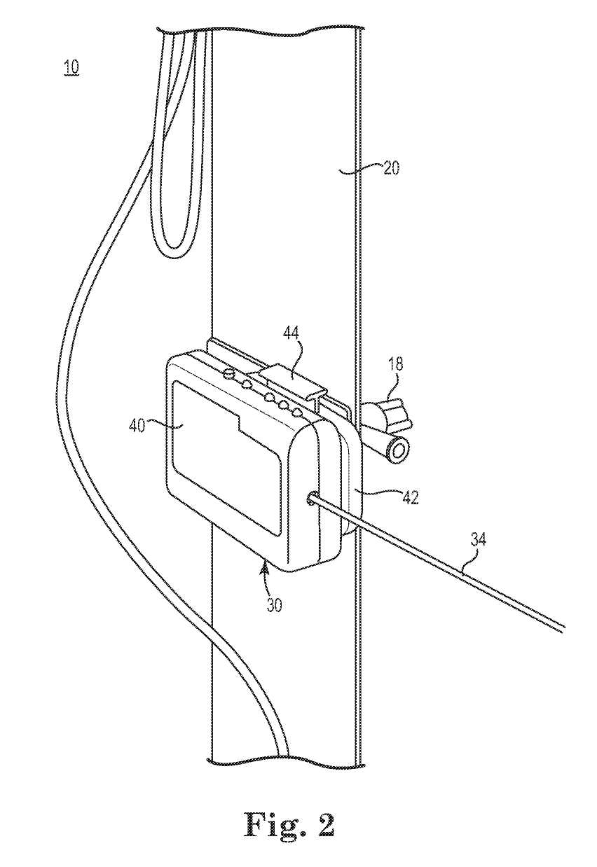

FIG. 2 is a rear view of a portion of drainage system 10 wherein laser leveling device 30 is rotatably mounted to panel 20. In particular, device 30 includes a housing 40 and a mounting bracket 42 coupled to the housing. Mounting bracket 42 includes a push button 44 for releasably securing mounting bracket 42 to the panel 20. Once mounting bracket 42 is secured to the panel 20, housing 40 can be rotated with respect to panel 20 and mounting bracket 42 so as to insure the housing 40 is level to gravity. Another example approach for mounting laser leveling device 30 to panel 20 is shown and described in U.S. Pat. No. 5,280,789; the contents of which are hereby incorporated by reference in their entirety. In any event, in one embodiment of laser leveling device 30, housing 40 is configured to rotate about mounting bracket 42 a minimum of 180°, such that laser beam 34 can be oriented in parallel, opposite directions when level with gravity. For example, with reference to FIG. 1, if patient 12 were located on an opposite side of panel 20, laser beam 34 can be oriented in an opposite direction to that shown in FIG. 1 (e.g., to the right).

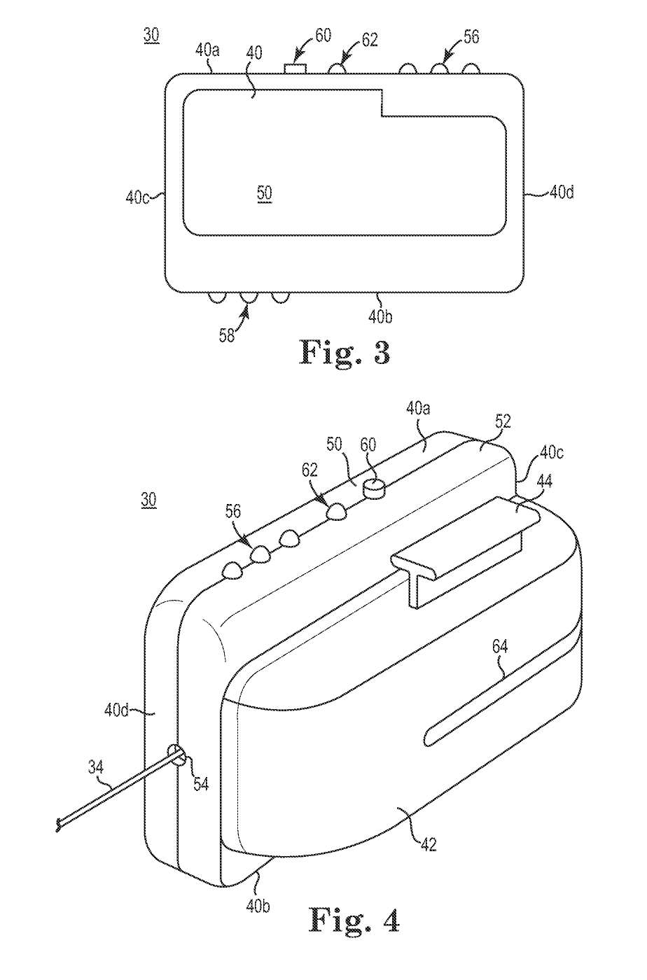

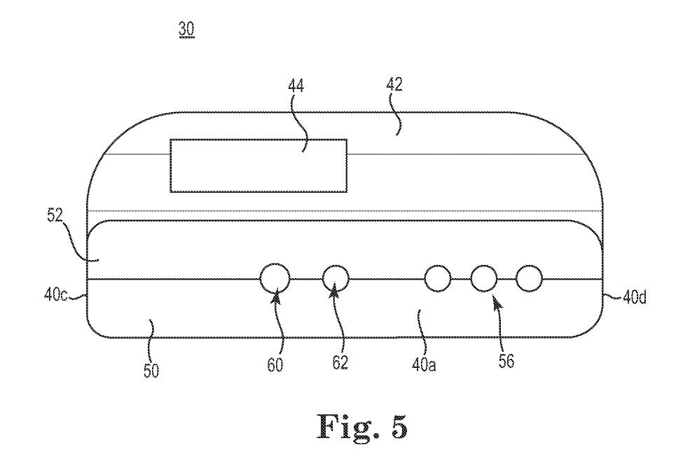

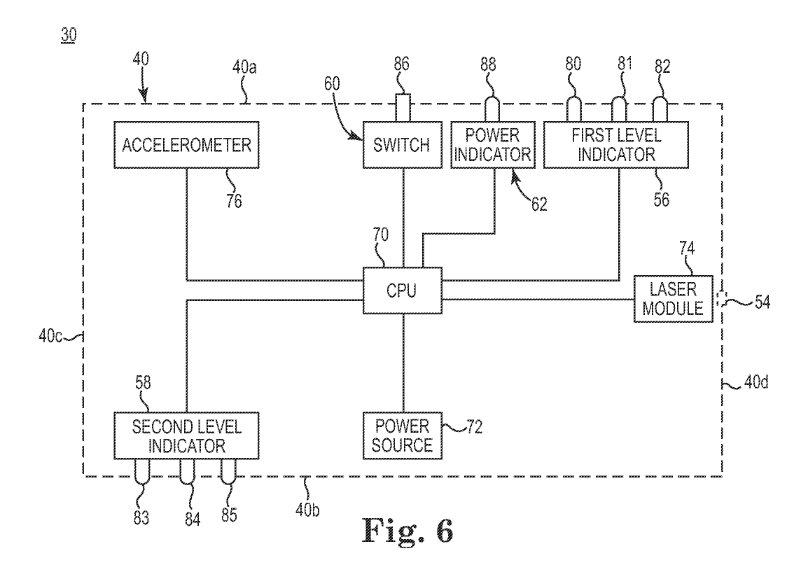

With reference to FIGS. 3-5, housing 40 is generally rectangular, defining a top 40a, bottom 40b and opposed sides 40c and 40d. Housing 40 further includes first and second housing portions 50 and 52 adapted to enclose electronic components of the laser leveling device 30, which are further described with respect to FIG. 6 below. Additionally, the housing 40 is configured to form openings for a laser aperture 54, a first level indicator 56, a second level indicator 58, a switch 60 and a power indicator 62. Laser aperture 54 is positioned on one side 40d of the housing 40 such that beam 34 is generally parallel to top 40a and bottom 40b. First level indicator 56 is positioned on the top 40a of the housing 40 whereas second level indicator 58 is positioned on the bottom 40b of the housing 40. As such, depending on a direction of orientation for laser beam 34, a level indicator will always be positioned in an upright fashion.

Each of the level indicators 56 and 58 are configured to provide a visual indication of whether housing 40 is level. In particular, the level indicator 56 and 58 provide an indication of whether top 40a, bottom 40b and laser beam 34 are parallel with respect to the ground. Switch 60 is provided to temporarily turn on the laser beam 34, wherein power indicator 62 provides an indication that power is provided to turn on the laser beam 34. In another embodiment, power indicator 62 can indicate a power level of a power source enclosed within housing 40 (e.g., battery level is low). Mounting bracket 42 further includes a groove 64 that cooperates with a tab on panel 20 of the drainage system 10 such that mounting bracket 42 is secured to panel 20.

Further reference is made to FIG. 6, which is a schematic block diagram of components at least partially enclosed within housing 40. In one embodiment, the components in FIG. 6 are electrically coupled together and mounted to a printed circuit board (not shown). As illustrated, first level indicator 56, second level indicator 58, switch 60 and power indicator 62 are partially exposed with respect to the housing 40 so as to provide visual and/or physical exterior exposure of the components. Enclosed within housing 40 are a CPU 70, a power source 72, a laser module 74 and an accelerometer 76. In one embodiment, CPU 70 is a MSP430™ microcontroller available from Texas Instruments of Dallas, Tex. Power source 72 can be a portable power source such as a battery and configured to provide power to each of the components in housing 40 and in particular, first level indicator 56, second level indicator 58, switch 60, power indicator 62, CPU 70, laser module 74 and accelerometer 76. Additionally, accelerometer 76 can be a three-axis accelerometer, for example part no. MMA7260QT from Freescale Semiconductor of Austin, Tex.

In the embodiment illustrated, first level indicator 56 includes three light emitting diodes (LEDs) 80, 81 and 82 that are exposed relative to the housing 40. Collectively, the LEDs provide an indication of whether the housing 40 is level with respect to gravity as a function of signals received from accelerometer 76. For example, outer LEDs 80 and 82 can be red LEDs whereas middle LED 81 is a green LED. If either LED 80 or LED 82 is lit, this is an indication that housing 40 is not level. If LED 81 is lit, this is an indication that housing 40 is level. In an alternative embodiment, first level indicator 56 can include a single LED indicating housing 40 is level. Second level indicator 58 is similarly constructed to first level indicator 56 and includes three LEDs 83, 84 and 85. Similar to LEDs 80-82, LEDs 83-85 can include two outer LEDS (e.g., LEDs 83 and 85) of a first color (e.g., red) and a middle LED (e.g., LED 84) of a second color (e.g., green) to indicate whether housing 40 is level. Switch 60 includes a push button 86 whereas power indicator 62 includes an LED 88 indicating whether the laser leveling device is operational.

To operate laser leveling device 30, push button 86 of switch 60 is actuated (i.e., depressed), which transmits a signal from switch 60 to CPU 70 indicating that the push button 86 has been depressed. CPU 70 then sends a signal to power indicator 62, laser module 74 and accelerometer 76 so as to turn on for a predetermined amount of time (e.g., approximately 20 seconds). During the predetermined amount of time, signals from accelerometer 76 are sent to CPU 70. CPU 70 interprets these signals and sends corresponding signals to first level indicator 56 and second level indicator 58. In particular, CPU 70 sends a signal to turn on one of the LEDs 80-82 of first level indicator 56 and one of the LEDs 83-85 of second level indicator 58. For example, if accelerometer 76 indicates that housing 40 is tilted in a clockwise direction (as viewed in FIG. 6), CPU 70 will send signals to turn on LEDs 82 and 85. Conversely, if accelerometer 76 indicates that housing 40 is tilted in a counter-clockwise direction (as viewed in FIG. 6), CPU 70 will send signals to turn on LEDs 80 and 83. If accelerometer 76 indicates that housing 40 is level, CPU 70 will send a signal to turn on LEDs 81 and 84. Once a user determines that housing 40 is level as indicated by LEDs 81 and 84, laser beam 34 can be aligned with zero reference point 32 (FIG. 1). To ensure a fixed relationship between laser module 74 and accelerometer 76, a calibration process can be utilized.

Although the present disclosure has been described with reference to preferred embodiments, workers skilled in the art will recognize that changes can be made in form and detail without departing from the spirit and scope of the present disclosure.

What is claimed is:

1. A laser leveling device for use with a patient drainage system, comprising:
a housing;
a mounting bracket coupled to the housing and configured to rotatably couple the housing to the drainage system;
an accelerometer enclosed within the housing;
a laser module enclosed within the housing; and
a level indicator electrically coupled to the accelerometer to indicate a relative position of the housing with respect to gravity.

2. The laser leveling device of claim 1, further comprising:
a power source enclosed within the housing to selectively provide power to the accelerometer, the laser module and the level indicator; and
a switch electrically coupled to the power source such that, upon actuation of the switch, the power source provides power to the accelerometer and the laser module for a predetermined period of time.

3. The laser leveling device of claim 1, wherein the level indicator includes a light emitting diode providing an indication that the housing is level with respect to gravity.

4. The laser leveling device of claim 3, wherein the laser leveling device includes three light emitting diodes, wherein two of the light emitting diodes indicate the housing is not level and one of the light emitting diodes indicate that the housing is level.

5. The laser leveling device of claim 1, further comprising:
a second level indicator electrically coupled to the accelerometer to indicate the relative position of the housing with respect to gravity.

6. The laser leveling device of claim 5, wherein the first-mentioned level indicator is positioned on a top of the housing and the second level indicator is positioned on a bottom of the housing.

7. The laser leveling device of claim 1 wherein the accelerometer is a three-axis accelerometer.

8. A drainage system, comprising:
a catheter configured to be fluidly coupled to a patient;
a drainage control component mounted to a panel spaced apart from the patient, the drainage control component fluidly coupled to the catheter;
a drip assembly slidably mounted to the panel for selective positioning with respect to the drainage control component and fluidly coupled to the drainage control component; and
a laser leveling device rotatably mounted to the panel, the laser leveling device comprising:
a housing;
an accelerometer enclosed within the housing;
a laser module enclosed within the housing; and
a level indicator electrically coupled to the accelerometer to indicate a relative position of the housing with respect to gravity.

9. The drainage system of claim 8, further comprising:
a power source enclosed within the housing to selectively provide power to the accelerometer, the laser module and the level indicator; and
a switch electrically coupled to the power source such that, upon actuation of the switch, the power source provides power to the accelerometer and the laser module for a predetermined period of time.

10. The drainage system of claim 8, wherein the level indicator includes a light emitting diode providing an indication that the housing is level with respect to gravity.

11. The drainage system of claim 10, wherein the laser leveling device includes three light emitting diodes, wherein two of the light emitting diodes indicate the housing is not level and one of the light emitting diodes indicate that the housing is level.

12. The drainage system of claim 8, further comprising:
a second level indicator electrically coupled to the accelerometer to indicate the relative position of the housing with respect to gravity.

13. The drainage system of claim 12, wherein the first-mentioned level indicator is positioned on a top of the housing and the second level indicator is positioned on a bottom of the housing.

14. The drainage system of claim 8 wherein the accelerometer is a three-axis accelerometer.

15. A method for draining fluid from a patient, comprising:
fluidly coupling a catheter to the patient;
fluidly connecting a drainage control component to the catheter;
positioning a laser leveling device in fixed relation to the drainage control component, the laser leveling device including a housing, an accelerometer, a laser module and a level indicator, wherein the level indicator is electrically coupled to the accelerometer to indicate a relative position of the housing with respect to gravity;
rotating the housing so as to be level with gravity;
powering the laser module to form a laser beam; and
aligning the laser beam with a reference point on the patient.

16. The method of claim 15, further comprising:
providing a power source enclosed within the housing; and
actuating a switch electrically coupled to the power source in order to provide power to the accelerometer and the laser module for a predetermined period of time.

17. The method of claim 15, further comprising:
powering a light emitting diode of the level indicator when the housing is level with respect to gravity.

18. The method of claim 15, further comprising:
powering a first light emitting diode when the housing is not level with respect to gravity; and
powering a second light emitting diode when the housing is level to gravity.

19. The method of claim 15, further comprising:
providing multiple level indicators electrically coupled to the accelerometer to indicate the relative position of the housing with respect to gravity.
